(19) United States
(12) Patent Application Publication
(10) Pub. No.: US 2016/0367683 A1
(43) Pub. Date: Dec. 22, 2016

(54) ENHANCING THE IN-VIVO EFFICIENCY OF PARTICLE DELIVERY THROUGH NON-COVALENT INTERACTION WITH RED BLOOD CELL SURFACE PROTEINS

(71) Applicant: South Dakota State University, Brookings, SD (US)

(72) Inventors: Hemachand Tummala, Brookings, SD (US); Pratik Muley, Brookings, SD (US)

(21) Appl. No.: 14/743,717

(22) Filed: Jun. 18, 2015

(51) Int. Cl. A61K 47/48; A61K 9/00; A61K 35/14; A61K 49/00

(52) U.S. Cl. CPC A61K 47/4813; A61K 49/0093; A61K 9/0019; A61K 35/14

(57) ABSTRACT

The present invention relates to the production of modified microscale and nanoscale particles that have enhanced in vivo delivery characteristics. The resultant particles can be used in any of a number of applications including informatics, detection, diagnosis, imaging, and/or therapeutics. Further, the modified particles of the present invention have enhanced circulation half time and elimination rate constant characteristics, and enhanced biodistribution.

### BACKGROUND OF THE INVENTION

Field of the Invention

The present invention relates to the field of particle technology. More particularly, the presently invention relates to microscale and nanoscale particle technology for in vivo delivery for informatics, detection, diagnosis, imaging, and/or therapeutics.

Background

Recent advancements in nanotechnology have unfolded novel opportunities in medicine, especially in targeted therapeutics and medical imaging. Encapsulation of active molecules in polymeric nanoparticles offers many advantages, including sustained release of the encapsulated drug, protection from degradation in circulation, and active or passive targeting to target tissues such as brain, liver or cancer tissue. In addition, polymeric nanoparticles have also been utilized as tools for imaging as well as contrasting agents. However, parenteral delivery of nanoparticles, especially through intravenous routes, poses major challenge mainly due to the rapid clearance of particles from the circulation. The circulating nanoparticles are quickly recognized by the Reticulo-Endothelial System (RES) following opsonization and are rapidly removed from circulation. Many particles (nano and micro) are cleared within a matter of minutes from circulation before reaching the target site and as a result their applicability is heavily dependent upon their ability to remain in the circulation for a reasonable period of time.

The importance of nanotechnology and nanocarrier-based treatments is particularly well exemplified in the context of cancer. Cancer, as a general disease, is the second most common cause of death in the U.S. Effective treatment of cancer requires a number of aspects, including informatics, detection, diagnostics, therapeutics, and imaging. All of these aspects could involve or be aided by nanotechnology by specifically targeting cancer. Such targeting can be passive or active, involving delivery of, for example, drugs, toxins, and nanodevices directly to the cancer cells. Such delivery preferably reduces or eliminates damage or toxicity to non-cancer cells; traditional cancer treatments like chemotherapy and radiation treatment are non-specific and induce damage to a wide variety of non-cancer cells and tissues.

A variety of nanocarriers exist, including nanotubes, micelles, liposomes, nanoscale polymeric carriers, protein carriers, carbohydrate carriers, neosomes, dendrimers, nanoshells, metal or semiconductor particles, and polymer-conjugate drugs/proteins. However, very limited success has been achieved using these existing nanocarriers for informatics, detection, diagnosis, imaging, and therapeutics. The limitations are largely attributable to the inability to translate the nanocarriers to a clinical setting due to the body detecting the nanocarriers as foreign entities, resulting in immune clearance, and/or removal by the RES system and liver.

Prior approaches to enhancing the efficacy of nanocarrier in vivo have focused primarily on reducing the body's ability to recognize the nanocarrier. Existing state-of art technology to prolong the circulation of the nanoparticles utilizes surface modification of the particles by chemical or physical attachment of polymers or proteins. These approaches have been limited in their efficacy, and are generally complicated. The use of polymers such as polyethylene glycols (PEG) and poloxamines results in increased circulation of nanoparticles; however, the circulation time is still significantly limited and could be further improved. In addition PEGylation (addition of PEG) has several disadvantages reported; PEGylation reduces the uptake of the nanocarriers by the target cells such as cancer cells, and also results in generation of anti-PEG antibodies after initial injection in some patients. These antibodies further enhance the clearance of PEGylated nanocarrier, and render them ineffective for further injections. Furthermore, the conjugation of the polymers to the nanoparticle surface is a cumbersome process, and also increases the hydrostatic diameter of the particles that could interfere with passive targeting of nanoparticles to more cryptic or difficult to access sites. This is because passive targeting depends on the size of the particles and as the size increases their ability to accumulate in tissue, such as cancer tissue, decreases. In addition, the use of monofunctional polymers (i.e. PEGylation) is expensive. Hence, there is a critical need for a novel, and yet simple strategy of enhancing the circulation of micro- and nanoparticles to replace the current technology.

The compositions and methods of the present invention provide for enhanced in vivo efficiency of particle delivery. The compositions and methods of the present invention for enhanced in vivo particle delivery utilize interactions with blood cells to mediate superior biodistribution. The methods and compositions provide higher efficiency than existing technologies, such as PEGylation, and avoid immune recognition and clearance. The compositions and methods of the enhanced in vivo particle delivery are useful for in vivo informatics, detection, diagnosis, imaging, and therapeutics. In particular, the methods and compositions are beneficial for diagnosis, treatment, and prevention of a variety of diseases and disorders, including but not limited to cancer.

It is thus an object of the present invention to provide methods and compositions for enhancing the in vivo efficacy of particle delivery. In one aspect, it is a further object to provide methods for producing microscale or nanoscale particles that reversibly bind to a protein or peptide on the surface of a blood cell through a molecule conjugated to the particle, thereby improving the circulation time and decreasing the elimination rate constant of the particles.

It is a further object of the present invention to provide methods for producing modified microscale or nanoscale particles with enhanced in vivo delivery.

It is a further object of the present invention to provide modified microscale or nanoscale particles with enhanced in vivo delivery.

It is a further object of the present invention to provide methods for diagnosing or treating diseases or conditions using the particles with enhanced in vivo delivery.

Other objects, advantages and features of the present invention will become apparent from the following specification taken in conjunction with the accompanying drawings.

### SUMMARY OF THE INVENTION

The present invention relates to compositions and methods for enhancing the in vivo efficiency of particle delivery. According to the invention, Applicants have developed systems and methods for direct interaction of nanocarriers with blood cells, resulting in enhanced circulation and biodistribution.

In one embodiment, the invention encompasses a method of enhancing the in vivo efficiency of particle delivery. The methods can comprise modifying the surface of a particle to allow interaction with a protein or peptide on the surface of a blood cell, thereby mediating enhanced circulation and biodistribution. In one aspect, the modification can be conjugation of the particle to a molecule that specifically binds to the protein or peptide on the surface of a blood cell. The conjugation can be covalent or non-covalent. The type of conjugation can be selected based on the molecule and the type of particle. The molecule conjugated to the particle is preferably non-toxic. In an exemplary embodiment, the molecule is glucose.

In a further aspect, the molecule conjugated to the particle reversibly binds to a protein or peptide on the surface of a blood cell. In one aspect, the protein or peptide can be a receptor, adhesion molecule, enzyme, channel, or transporter protein. In an exemplary embodiment, the protein or peptide is a GLUT1 transporter.

In another aspect, the particles of the present invention are nanoparticles. The nanoparticles can be, for example, nanoshells, liposomes, neosomes, protein particles, polymeric carriers, protein carriers, carbohydrate carriers, neosomes, dendrimers, micells, carbon nanotubes, semiconductor or metal particles, and polymers-conjugate drug constructs.

In a further aspect, the blood cell to which the modified microscale or nanoscale particles interact are one or more type of blood cell, including include red blood cells (RBCs), leukocytes, and platelets. In an exemplary embodiment, the cells are RBCs.

In another embodiment, the present invention provides modified microscale or nanoscale carrier assembly compositions comprising a particle and a molecule conjugated to the surface of said particle, wherein said molecule reversibly binds to a protein or peptide on the surface of a blood cell. In one aspect, the modified carrier assembly comprises a nanoshell, liposome, polymeric carrier, dendrimer, micell, carbon nanotube, semiconductor or metal particle, or polymers-conjugate drug construct. In a further aspect, the molecule conjugated to the particle reversibly binds to a protein or peptide on the surface of a blood cell. In one aspect, the protein or peptide can be a receptor, adhesion molecule, enzyme, channel, or transporter protein. In an exemplary embodiment, the protein or peptide is a GLUT1 transporter.

In another embodiment, the present invention provides methods of diagnosing or treating a disease comprising administering to an individual a modified microscale or nanoscale carrier assembly comprising a microscale or nanoscale particle, and a molecule conjugated to the surface of said particle, wherein said molecule reversibly binds to a protein or peptide on the surface of a blood cell. In one aspect, the methods involve molecules conjugated to the surface of particle that bind to a protein or peptide on the surface of a red blood cell. In a further aspect, the methods involve using microscale or nanoscale particles having enhanced circulation half time (T_{1/2}) and/or reduced elimination rate constant (kₑₗ). In a preferred embodiment, the T_{1/2} of the modified particles is greater than about 20 minutes. In a more preferred embodiment, the T_{1/2} is greater than about 30 minutes, more preferably greater than about 60 minutes, more preferably greater than 85 minutes, more preferably greater than about 90 minutes. In an exemplary embodiment, the T_{1/2} is greater than about 120 minutes. In another preferred embodiment, the kel of the modified particles is less than about 0.05/hr. In a more preferred embodiment, the kel is less than about 0.025/hr, more preferably less than about 0.01/hr, and even more preferably less than about 0.0075/hr. In an exemplary embodiment, the kₑₗ of the modified particles is less than 0.006/hr.

In a further aspect, methods of treating or diagnosing can involve administration of the modified particles to an individual or subject in need thereof. In one embodiment, administration can be through parenteral routes, such as intravenous injection. In another embodiment, administration can be through injection into any circulation system, including the lymphatic system. In a different embodiment, administration may be obtaining a blood sample from said individual, combining said modified microscale or nanoscale carrier assembly with said blood sample, and introducing said blood sample with said modified microscale or nanoscale carrier assembly to the individual through parenteral routes, such as intravenous injection.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### DESCRIPTION OF THE FIGURES

FIG. 1 shows a schematic of an approach for enhancing in vivo efficiency of particle delivery according to an exemplary embodiment of the invention.

FIG. 2 shows the physicochemical characteristics of nanoparticles that are unmodified or surface modified with fructose, glucose, or PEG according to an exemplary embodiment of the invention.

FIG. 3 shows in vitro binding of glucose modified nanoparticles with human red blood cells, according to an exemplary embodiment of the invention.

FIG. 4 shows in vitro binding of nanoparticles to red blood cells in the presence of serum.

FIG. 5 shows the dependence of glucose modified nanoparticles to RBCs is due to specific interaction of glucose with the GLUT1 transporter on the surface of RBCs.

FIG. 6 (A-B) shows that attachment of glucose modified nanoparticles to RBCs according to an exemplary embodiment of the invention is reversible under shear stress. (A) shows a schematic of detachment of nanoparticles from RBCs by inducement of shear stress. (B) shows the amount of detachment of nanoparticles from RBCs under increasing shear stress.

FIG. 7 (A-B) shows that nanoparticles reattach to RBCs according to an exemplary embodiment of the invention following detachment by shear stress. (A) shows a schematic of attachment of nanoparticles to RBCs, detachment by shear stress, and reattachment of the nanoparticles to RBCs. (B) shows the amount of nanoparticles that reattach to RBCs following detachment.

FIG. 8 shows a lack of hematoxicity of glucose modified nanoparticles according to an exemplary embodiment of the invention.

FIG. 9 shows a schematic of in vivo circulation studies using unmodified nanoparticles or nanoparticles surface modified with glucose, fructose, or PEG according to an exemplary embodiment of the invention. The nanoparticles were administered intravenously, and biodistribution and circulation time in blood were subsequently determined.

FIG. 10 shows the circulation time of nanoparticles modified according to an exemplary embodiment of the invention. The time until 50% of nanoparticles (T₅₀) and 10% (T₁₀) were remaining in circulation was determined.

FIG. 11 shows tissue distribution of unmodified nanoparticles or nanoparticles modified with fructose, glucose, or PEG 24 hours after intravenous administration.

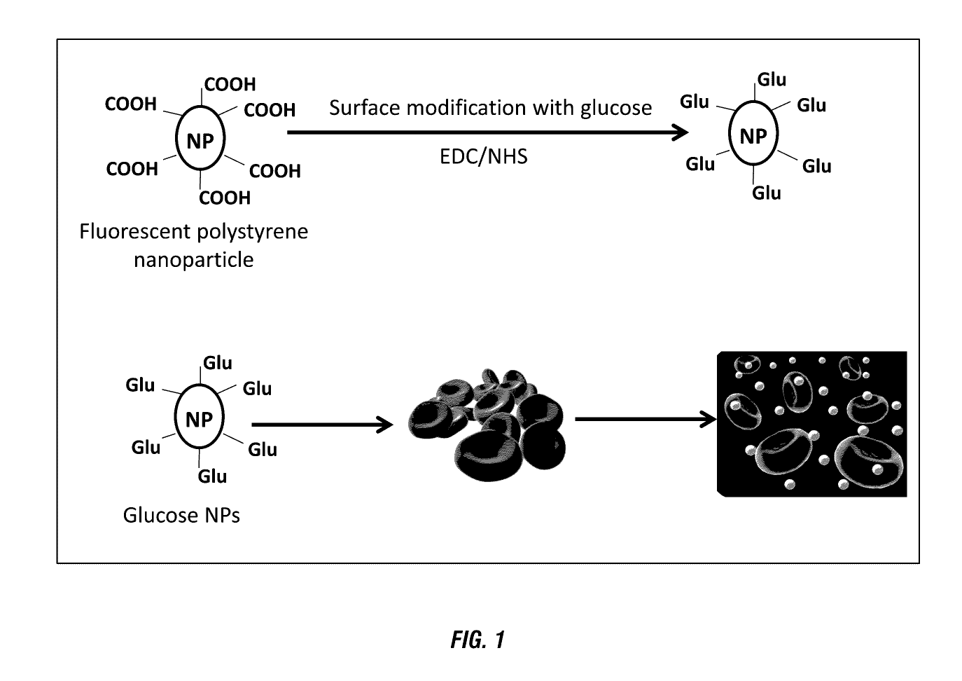

Various embodiments of the present invention will be described in detail with reference to the drawings, wherein like reference numerals represent like parts throughout the several views. Reference to various embodiments does not limit the scope of the invention. Figures represented herein are not limitations to the various embodiments according to the invention and are presented for exemplary illustration of the invention.

### DETAILED DESCRIPTION

Unless otherwise defined herein, scientific and technical terms used in connection with the invention shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well known and commonly used in the art. The methods and techniques are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol. I, CRC Press (1976); Handbook of Biochemistry: Section A Proteins, Vol. II, CRC Press (1976); Essentials of Glycobiology, Cold Spring Harbor Laboratory Press (1999), which are incorporated herein by reference.

The following terms, unless otherwise indicated, shall be understood to have the following meanings:

The articles “a” and “an” are used herein to refer to one or to more than one (i.e., to at least one) of the grammatical object of the article. By way of example, “an element” means one element or more than one element.

As used herein, the term “agglomeration” refers to the formation of an aggregate (a cohesive mass consisting of particulate subunits) in a suspension through physical (van der Waals or, hydrophobic) or electrostatic forces. The resulting structure is called an “agglomerate.”

As used herein, “blood cells” refers to cells that are commonly found in the circulatory system. Blood cells include red blood cells (RBCs), leukocytes, and platelets.

The terms “conjugate,” “conjugated,” and “conjugating” refer to the formation of a bond between molecules, an in particular between a molecule and the surface of a particle. The molecule that is conjugated to the particle is referred to as the “conjugate molecule”. In particular, the conjugate molecule interacts or binds to a protein or peptide on the surface of a blood cell, thereby mediating interaction between a particle and the blood cell. Conjugation can be direct (i.e. a bond between a molecule and the surface of a particle) or indirect (i.e. between the conjugate molecule and a second molecule that is already bound to the surface of a particle). The conjugation can be covalent or non-covalent.

The term “crosslink” refers to a bond or chain of atoms attached between and linking two different molecules, such as polymer chains.

As used herein, non-aggregating is the state of “dispersed” bioparticulates.

In the present invention, an “effective amount” or “therapeutically effective amount” of a compound or of a composition of the present invention is that amount of such compound and/or composition that is sufficient to effect beneficial or desired results as described herein. In terms of treatment of a mammal, e.g., a human patient, an “effective amount” is an amount sufficient to treat, reduce, manage, palliate, ameliorate, or stabilize a condition, such as a non-congenital oncosis or extended quiescence of the cells of a mammal, or both, as compared to the absence of the compound or composition.

“Enriching,” as the term is used herein, refers to the process by which the concentration, number, or activity of something is increased from a prior state. For example, a population of 100 leukocytes is considered to be “enriched” in leukocytes if the population previously contained only 50 leukocytes. Similarly, a population of leukocytes is also considered to be “enriched” in leukocytes if the population previously contained 99 leukocytes. Likewise, a population of 100 leukocytes is also considered to be “enriched” in leukocytes even if the population previously contained zero leukocytes.

The term “hydrogel” refers to a water-swellable polymeric matrix, consisting of a three-dimensional network of macromolecules held together by covalent crosslinks that can absorb a substantial amount of water to form an elastic gel.

As the term is used herein, “isolated” refers to a polynucleotide, polypeptide, protein, molecule, compound, material or cell of genomic or synthetic origin or some combination thereof which is not associated with all or a portion of the polynucleotides, polypeptides, proteins, molecules, compounds, materials or cells with which the isolated polynucleotide, polypeptide, protein, molecule, compound, material or cell is found in nature, or is linked to a polynucleotide, polypeptide, protein, molecule, compound, material or cell to which it is not linked in nature.

As used herein, the terms “nanoscale” and “nanosize” refer to a special state of subdivision implying that a particle has an average dimension smaller than approximately 300 nm and exhibits properties not normally associated with the bulk phase, e.g., quantum optical effects.

The term “non-toxic” as used herein refers to molecules and substances, molecules, etc. to which the body does not exhibit an adverse reaction; the body exhibits a tolerance; and/or does not induce cell death or damage in the body under normal conditions. The term includes, for example, substances and molecules that are considered vitamins, nutrients, carbohydrates, cytokines, chemokines, carriers, salts and the like. The term also encompasses homologues, analogues, and molecular mimics of such substances.

“Non-covalent” refers to any molecular interactions that are not covalent—i.e. the interaction does not involve the sharing of electrons. The term includes, for example, electrostatic, π-effects, van der Waals forces, and hydrophobic effects. “Covalent” refers to interactions involving the sharing of one or more electrons.

“Particle” as used herein refers to micro- and nanoscale assemblies and constructs. The term includes, but is not limited to, nanoshells, liposomes, polymeric carriers, protein carriers, carbohydrate carriers, neosomes, dendrimers, micells, carbon nanotubes, polymers-conjugate drug constructs. The term also includes microscale and nanoscale carriers.

The term “PEG” as used herein refers to poly(ethylene glycol).

As the term is used herein, “population” refers to two or more cells.

“Reversible” in relation to binding refers generally to interactions between molecules that do not involve covalent bonding. Reversible binding, therefore, does not require the cleavage of a covalent bond for the binding to be broken.

“Substantially homogeneous,” as the term is used herein, refers to a population of a substance, material, cell, etc. that is comprised primarily of that substance, material, cell, etc., and one in which impurities have been minimized.

As the term is used herein, “substantially separated from” or “substantially separating” refers to the characteristic of a population of first substances being removed from the proximity of a population of second substances, wherein the population of first substances is not necessarily devoid of the second substance, and the population of second substances is not necessarily devoid of the first substance. However, a population of first substances that is “substantially separated from” a population of second substances has a measurably lower content of second substances as compared to the non-separated mixture of first and second substances.

In one aspect, a first substance is substantially separated from a second substance if the ratio of the concentration of the first substance to the concentration of the second substance is greater than about 1. In another aspect, a first substance is substantially separated from a second substance if the ratio of the concentration of the first substance to the concentration of the second substance is greater than about 2. In yet another aspect, a first substance is substantially separated from a second substance if the ratio of the concentration of the first substance to the concentration of the second substance is greater than about 5. In another aspect, a first substance is substantially separated from a second substance if the ratio of the concentration of the first substance to the concentration of the second substance is greater than about 10. In still another aspect, a first substance is substantially separated from a second substance if the ratio of the concentration of the first substance to the concentration of the second substance is greater than about 50. In another aspect, a first substance is substantially separated from a second substance if the ratio of the concentration of the first substance to the concentration of the second substance is greater than about 100. In still another aspect, a first substance is substantially separated from a second substance if there is no detectable level of the second substance in the composition containing the first substance.

### Enhanced Particle Delivery Assemblies and Systems

In one embodiment, the present invention provides systems for enhanced delivery of particles to organs, tissues, and other locations in the body. In one aspect, particles in the system can include micro- and nanoparticles, such as nanoshells, liposomes, polymeric carriers, protein carriers, carbohydrate carriers, neosomes, dendrimers, micells, carbon nanotubes, semiconductor or metal particles, and polymers-conjugate drug constructs. The particles can be conjugated to one or more molecules that mediate binding to a surface protein on a cell or other component of the circulatory system. In a preferred embodiment, the particle is conjugated to a molecule that mediates binding to a red blood cell.

In one exemplary embodiment, glucose is non-covalently conjugated to the surface of a particle and reversibly binds to GLUT1 glucose transporter molecules on the surface of RBCs when administered to an individual. The binding of glucose to GLUT1 mediates the interaction of the particle with the RBC, which in turn results in the RBC carrying the particle through the individual's circulatory system. The reversibility of glucose binding to GLUT permits the particle to be released or separated from the RBC under conditions of shear stress, for example in a capillary or other physiologic high-shear setting or any other physiological intervention. The interaction of the particle with the blood cell, mediated by the surface conjugation of the particle, results in increased circulation time, increased biodistribution, and decreased elimination from the body, compared to non-modified particles.

### Conjugate Molecules

Molecules useful for conjugation to particles according to the present invention are generally non-toxic molecules that can facilitate interaction with a blood cell. The conjugate molecules can be conjugated to particles via covalent or non-covalent interactions. The conjugate molecules can interact with blood cells through covalent or non-covalent binding of surface proteins. Conjugate molecules are preferable but not limited to small molecules in order to prevent the particle-conjugate molecule complex from becoming too large to be effectively transported by circulation.

According to one aspect of the invention, conjugate molecules can reversibly bind to surface proteins or peptides on blood cells. In a further aspect, the proteins or peptides to which the conjugate molecules bind will generally be present on the surface of a cell and remain on the surface of the cell during circulation. The proteins and peptides can be, for example, receptors, adhesion molecules, molecules from cyto-skeleton structure, enzymes, channels, and/or transporter proteins. In a preferred embodiment, the surface protein specifically binds the conjugate molecule. In one aspect, the conjugate molecule is a ligand or substrate of the surface protein. In another aspect the conjugate molecule can be a homologue or mimic of the ligand or substrate of the surface protein. The binding could be specific as exemplified by glucose to GLUT1 transporters or non-specific to unknown molecules on the surface of the blood cells.

### Particles

The present invention can utilize a variety of microscale and nanoscale particles. Generally, particles useful for the present invention are designed or adapted to use in informatics, detection, diagnosis, imaging, and/or therapeutics. Particles can include, for example, hydrogels, nanoshells, liposomes, polymeric carriers, protein carriers, carbohydrate carriers, neosomes, dendrimers, micells, carbon nanotubes, semiconductor or metal particles, and polymers-conjugate drug constructs.

In one aspect, particles included in the present invention can be nanoshells. Nanoshells are composed of a shell made of a conducting material such as a metal, and a core composed of a non-conducting material. The nanoshells can be 5-500 nm in diameter, preferably about 10 to about 300 nm, and more preferably about 15 to about 200 nm. The outer shell layer of the nanoshells can have a thickness in the range between about 1 nm and about 100 nm. The nanoshells can be any shape such as, without limitation, spherical, rod or fiber shaped. Generally, nanoshells having a relatively low polydispersity are preferred. In a non-limiting example, a nanoshell may be composed of a shell with a core diameter in the range of about 50 nm to about 200 nm with a gold shell having a thickness ranging from about 5 nm to about 25 nm. The core material can be, for example, dielectric materials or semiconductor materials. Exemplary but non-limiting core materials include colloidal silica, silicon dioxide, titanium dioxide, polymethyl methacrylate (PMMA), polystyrene, and gold sulfide, and semiconductor materials such as, without limitation, CdSe, CdS, or GaAs. The shell material is preferably a conducting material, such as a metal, e.g. without limitation, the noble metals and coinage metals, or an organic conducting material such as polyacetylene and doped polyanaline. More specifically the shell may include, but is not necessarily limited to, metals such as gold, silver, copper, platinum, palladium, lead, iron, biodegradable metals such as magnesium, zinc, calcium, or tungsten, and alloys and combinations thereof.

In another aspect, particles included in the present invention can include liposomes. Liposomes as used herein include any artificially-prepared spherical vesicle composed of a lamellar phase lipid bilayer. Liposomes also include lipid coated micro- or nanoparticles. The interior of a liposome is typically an aqueous environment, and it may comprise an agent such as but not limited to a prophylactic, therapeutic or diagnostic agent. In some instances, the liposomes do not comprise a solid core, such as a solid polymer core (e.g., a synthetic polymer core). Instead, the liposomes may have a fluid core comprising agents (i.e. substances, drugs, etc.) for delivery to a particular site in the body. The agents are typically included in the lipid solution during the synthesis process and in this manner are incorporated (e.g., by encapsulation) into the liposomes during synthesis. Lipophilic molecules may also be incorporated directly into the lipid bilayers as the liposomes are formed or molecules with lipophilic tails may be anchored to the lipid bilayers during liposome formation. The liposomes may be produced in the absence of harsh solvents, such as organic solvents, and as a result they may be able to encapsulate a wide variety of agents including those that would be susceptible to organic solvents and the like. Particles used in the present invention may also include micells. Micelles are lipid molecules that arrange themselves in a spherical form in aqueous solutions.

In another aspect, particles included in the present invention can include polymer nanocomposites. Polymer nanocomposites include polymer nanocarriers and polymer nanoparticles. Polymer nanocomposites generally consist of a polymer or copolymer having nanoparticles or nanofillers dispersed in the polymer matrix. These may be of different shape (e.g., platelets, fibers, spheroids), but at least one dimension is in the range of 1-50 nm. Polyer nanocomposites also include bio-hybrid polymer nanocarriers and nanoparticles wherein biological objects such as protein are immobilized on a nanocomposite, usually by adsorption or by chemical binding and to a lesser extent by incorporating these objects as guests in host matrices.

In another aspect, particles included in the present invention can include dendrimers. Dendrimers are defined by regular, highly branched segments leading to a relatively monodisperse, tree-like or generational structure. Dendrimers possess three distinguishing architectural features: the core; the interior area containing branch upon branch of repeat units or generations with radial connectivity to the core; and an exterior or surface region of terminal moieties attached to the outermost generation. A dendrimer can be defined into a multitude of structures by tuning these three architectural components. Dendrimers that are highly branched and reactive three-dimensional macromolecules have become increasingly important in biomedical applications due to their high degree of molecular uniformity, narrow molecular weight distribution, specific size and intriguing structural properties such as internal voids and cavities, and a highly functional terminal surface. The spatially arranged functional groups can react with a variety of molecules, for example, hydrophilic molecules such as PEO (polyethylene oxide or PEG) to increase their blood circulation times, contrast agents for use in magnetic resonance imaging (MRI), and targeting molecules to localize to desired tissue sites.

Currently available dendrimers contain benzyl ether, propyleneimine, amidoamine, L-lysine, ester and carbosilane dendritic segments. Among them, cationic polyamidoamine (PAMAM) dendrimers have been widely studied and were reported to mediate high levels of gene transfection in a wide variety of cells, depending on the dendrimer-DNA ratio, the size and especially the flexibility of the dendrimers. PAMAM dendrimers are considered targeted delivery systems, and can enhance accumulation within certain tumor microvasculature, increase extravasation into tumor tissue. Poly(L-lysine) (PLL) dendrimer is another polycationic dendrimer containing a large number of surface amines and considered to be capable of the electrostatic interaction with polyanions, such as nucleic acids, proteoglycans found in extracellular matrix and phospholipids of the cell membrane. These polymers can localize drugs, including lipid-derived bioactive growth arresting, pro-apoptotic metabolites or agents to the targeted membranes.

However, polycationic dendrimers still have in vivo toxicity problems and are resistant to degradation in the body and are thus less suitable for drug delivery. To improve the cytotoxicity of PAMAM dendrimers, the cationic amine terminal groups of the dendrimers can be replaced with anionic carboxylate terminal groups. The present inventive materials address some of the disadvantages of dendrimer structures prepared from individual components by combining smart and degradable segments as arms, branches, or dendrons of a dendrimeric structure. Such dendrimeric materials can be prepared by coupling a thermoresponsive polymer segment with a biodegradable polymer segment in a chemical bond forming reaction.

Dendrimers also can be prepared as nano-sized particles. It is believed that particles having a size of about 1 nm to 1000 nm hold a significant advantage in transporting and targeting drugs to inflamed, proliferative or transformed tissues. Drugs are loaded into the nano-sized dendrimers by adsorption, entrapment and covalent attachment, and released from the nano-sized dendrimers by desorption, diffusion, polymer erosion or some combination of any or all the above mechanisms. In vitro and in vivo experiments show that nano-sized dendrimers can have long blood circulation times and a low RES uptake when they are stabilized by dextran and coated with polysorbate 80. The nano-sized dendrimers may be able to interact with the blood vessel or solid tumor cells, and then be taken up by these cells by endocytosis. Dendrimers are believed, therefore, to have a great potency to deliver drugs to tumorigenic or inflamed/proliferative tissues due to increased circulatory half-life.

### Cell Types

According to one aspect, particles interact with blood cells via the conjugate molecules conjugated to the particles. The selection of the particular cell type to which the particle is conjugated will depend at least in part on the intended target for the particle. In a preferred embodiment, the particle is conjugated to an RBC through one or more surface proteins on the RBC. In a more preferred embodiment the particle is conjugated to an RBC through binding of glucose to the GLUT1 transporter on the surface of the RBC.

In another embodiment, targeting of locations within or adjacent to the lymphatic system may require conjugation to cells that enter that portion of the circulatory system. For example, lymphocytes such as T and B cells may be selected for conjugation. A person of skill in the art would understand how to select appropriate proteins on the surface of lymphocytes to which to conjugate the particle.

### Methods of Enhancing Efficiency of In Vivo Particle Delivery

In one embodiment, the present invention provides methods of enhancing the efficiency of in vivo particle delivery comprising. The methods involve conjugating molecules to the surface of particles to produce surface modified particles. The particles can be a microscale or nanoscale particles. In one aspect, the particles can be a nanoshells, liposomes, polymeric carriers, protein carriers, carbohydrate carriers, neosomes, dendrimers, micells, carbon nanotubes, semiconductor or metal particles, or polymers-conjugate drug constructs. In a preferred embodiment, the particles are nanoscale.

In another aspect, the molecules for conjugation to the particles in the methods of the invention bind to a protein or peptide on the surface of a blood cell. In a preferred embodiment, the molecule reversibly binds to the protein or peptide. The protein or peptide can be expressed on the surface of one or more types of blood cells. In a preferred embodiment, the blood cell is a red blood cell. In another preferred embodiment, the surface protein or peptide on the blood cell is a receptor, adhesion molecule, enzyme, channel, or transporter protein. In an exemplary embodiment, the protein or peptide is the GLUT1 transporter. In another preferred embodiment, the molecule conjugated to the particle is a non-toxic small molecule. Such small molecules are preferably sufficiently small as to not adversely affect the circulate half time and/or elimination rate constants of the particle to which they are conjugated. In an exemplary embodiment, the non-toxic small molecule is glucose.

The methods also involve conjugation of the molecule to the particle. Conjugation can be accomplished using techniques known in the art, and should be selected based on the type of particle and the particular molecule to be conjugated. The conjugation can be covalent or non-covalent, and is preferably non-covalent. Conjugation can be accomplished by using varying chemical groups such as, for example, alcohols, carboxylic acids, amines, aldehydes, acid chlorides, anhydrides, or thiols. In some aspects, the chemical group can be a reactive carboxylic acid, such as aspartic acid, and glutamic acid. In other aspects, the chemical group can be a reactive amine, such as lysine. In other aspects, the chemical group can be a reactive thiol, such as cysteine. In other aspects, conjugation can be accomplished using, for example, enzymatic incorporation, biotinylation, thioether linkage, disulfide linkage, ester linkages, hydrazide linkages, amide linkages. In an exemplary embodiment, conjugation can be accomplished through crosslinking, such as amine coupling through 1-Ethyl-3-(3-dimethylaminopropyl)-carbodiimide/N-hydroxysuccinimide (EDC/NHS) coupling.

### Methods of Delivering Particles In Vivo

In one embodiment, the present invention provides methods for enhanced delivery of particles in vivo. In one aspect the methods involve introducing a particle to an individual, wherein the particle has been conjugated to a molecule that mediates binding to a protein or peptide on the surface of a blood cell.

In a further aspect, the method can involve administration to an individual by various routes. Administration can be local, regional, systemic, or continual administration. Administration can be by any appropriate route, for example by the oral (including buccal or sublingual), rectal, nasal, topical (including buccal, sublingual, or transdermal), vaginal, or parenteral (including subcutaneous, intracutaneous, intramuscular, intra-articular, intrasynovial, intrasternal, intrathecal, intralesional, intravenous, or intradermal injections or infusions) route.

In another aspect, the methods can involve administration by combining a blood sample or isolated blood cells from an individual with surface modified microscale or nanoscale particles, allowing the modified microscale or nanoscale particles to bind to the isolate blood cells or blood cells in the blood sample, and introducing the blood sample or isolated blood cells bound to said particles to the individual.

The methods of the present invention can be used to treat or diagnose a disease in a subject in need thereof. The methods involve administering a modified microscale or nanoscale particle to a person or subject in need of treatment or diagnosis. The particle can be any particle designed or adapted to use in informatics, detection, diagnosis, imaging, and/or therapeutics. Particles can include, for example, nanoshells, liposomes, polymeric carriers, protein carriers, carbohydrate carriers, neosomes, dendrimers, micells, carbon nanotubes, semiconductor or metal particles, and polymers-conjugate drug constructs. In one aspect, the modified particles are used to enhance delivery of a drug or therapeutic compound to a particular area or site in the body. In an exemplary embodiment, the modified particles are used to enhance delivery to a cancer or tumor site in the body. In another aspect, the modified particles can be used to enhance delivery of particles, or chemical or compounds carried by the particles, used in diagnostic imaging, such as magnetic resonance imaging (MRI), computed tomography (CT), positron emission topography (PET), and fluorescence imaging. Examples of particles useful for such methods include, but are not limited to, metal or semiconductor particles, nanoshells with dielectric or semiconductor cores, fluorescent dyes and other fluorophores, contrast agents, paramagnetic ansd superparamagentic materials, radiotracers, and semiconductor quantum dots.

The methods further involve increasing the circulation half time (T_{1/2}) of the modified microscale or nanoscale particle and/or decreasing the elimination rate constant (kₑₗ) of the modified microscale or nanoscale particles, relative to non-modified particles of the same type. In a preferred embodiment, the T_{1/2} of the modified particles is greater than about 20 minutes. In a more preferred embodiment, the T_{1/2} is greater than about 30 minutes, more preferably greater than about 60 minutes, more preferably greater than about 90 minutes. In an exemplary embodiment, the T_{1/2} is greater than about 120 minutes. In another preferred embodiment, the kₑₗ of the modified particles is less than about 0.05/hr. In a more preferred embodiment, the kₑₗ is less than about 0.025/hr, more preferably less than about 0.01/hr, and even more preferably less than about 0.0075/hr. In an exemplary embodiment, the kₑₗ of the modified particles is less than 0.006/hr.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1

### Glucose Modification of Nanoparticles Mediates Binding to RBCs

Although a plethora of literature exists on nanocarriers, very limited success has been achieved with respect to effective delivery by nanocarriers, particularly to target tissues such as brain, liver or cancer tissue. The most important reason for this inability to translate nanocarrier success in clinical setting is that the body detects nanoparticles as foreign and rapidly clears them out.

The Inventors have developed a novel approach to enhance the circulation time of polymeric nanoparticles by modifying them to attach to the surface of Red blood cells (RBCs) in-situ (inside the body). In this approach, nanoparticles are surface conjugated with glucose and are allowed to attach to RBCs through glucose transporter GLUT1, which is abundantly present on the outer membrane of RBCs. Results indicate that the glucose modified nanoparticles are capable of binding to the RBCs even in the presence of serum and other biological components of whole blood. We have also shown that that interaction between the nanoparticles and RBCs is non-covalent and reversible. This is important because, otherwise, the nanoparticles will never leave RBCs to reach their target site. The novel surface-modified nanoparticles were found to exhibit enhanced circulation time greater than that of poly-ethelene glycol (PEG) modified nanoparticles. Furthermore, this approach can be applied to any particulate delivery system including metallic particles, liposomes, dendrimers and micelles to enhance their circulation.

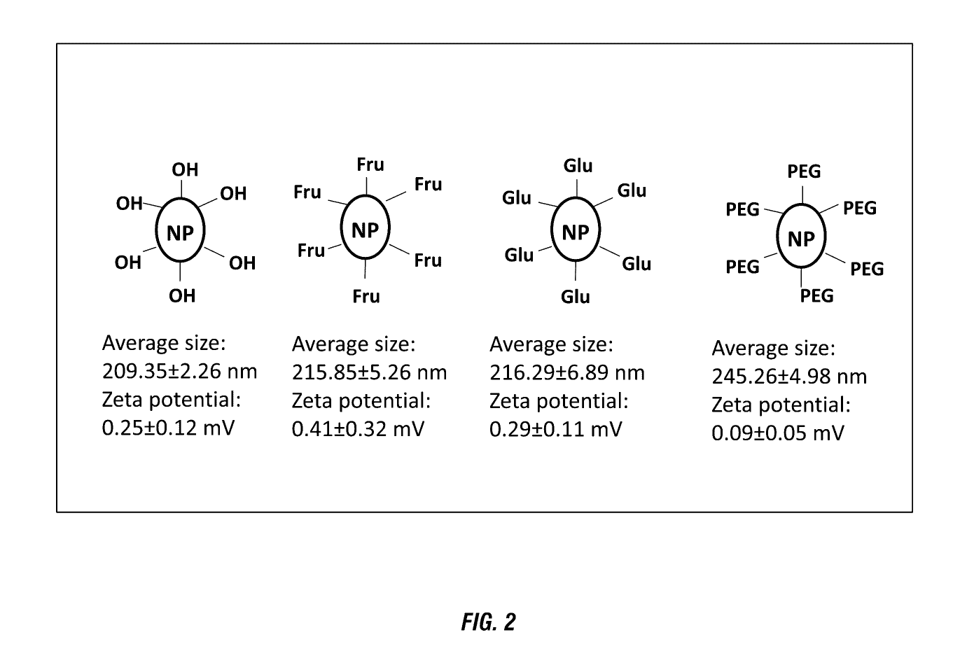

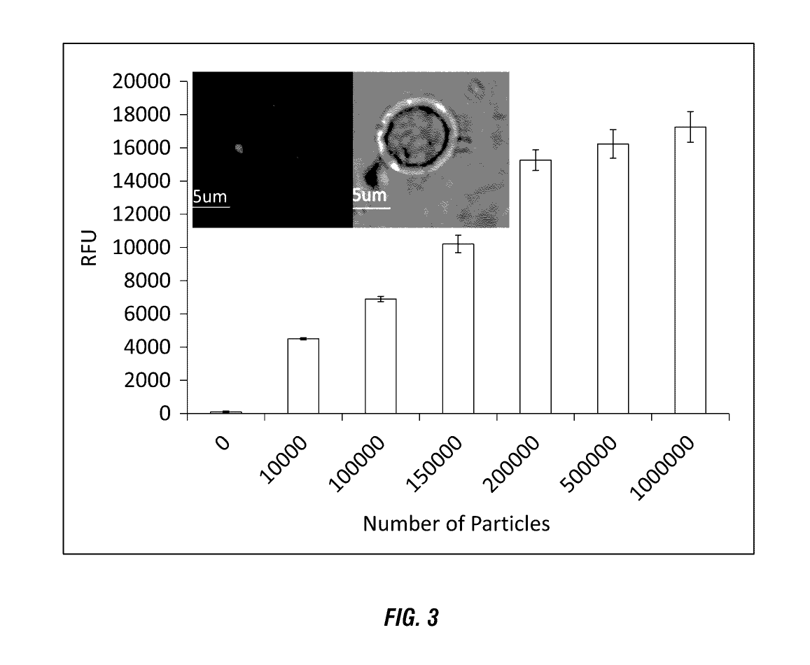

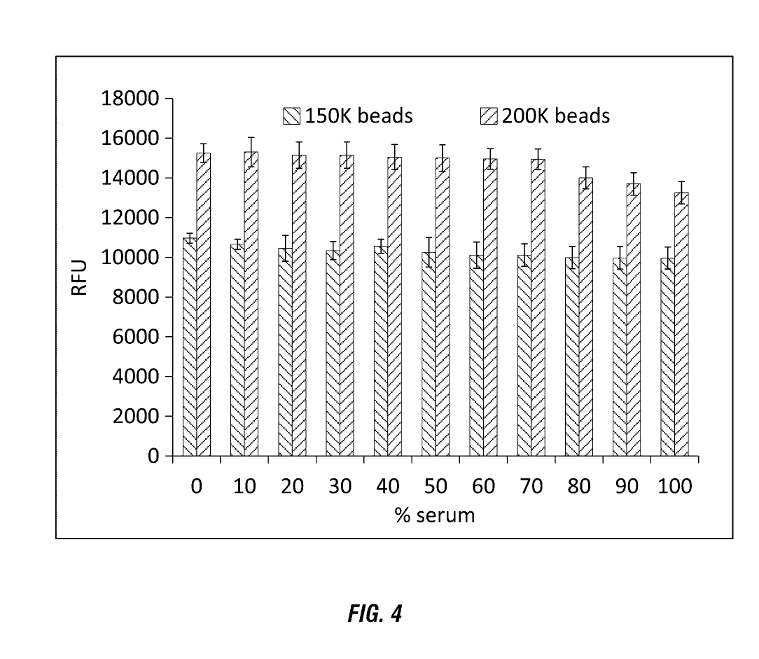

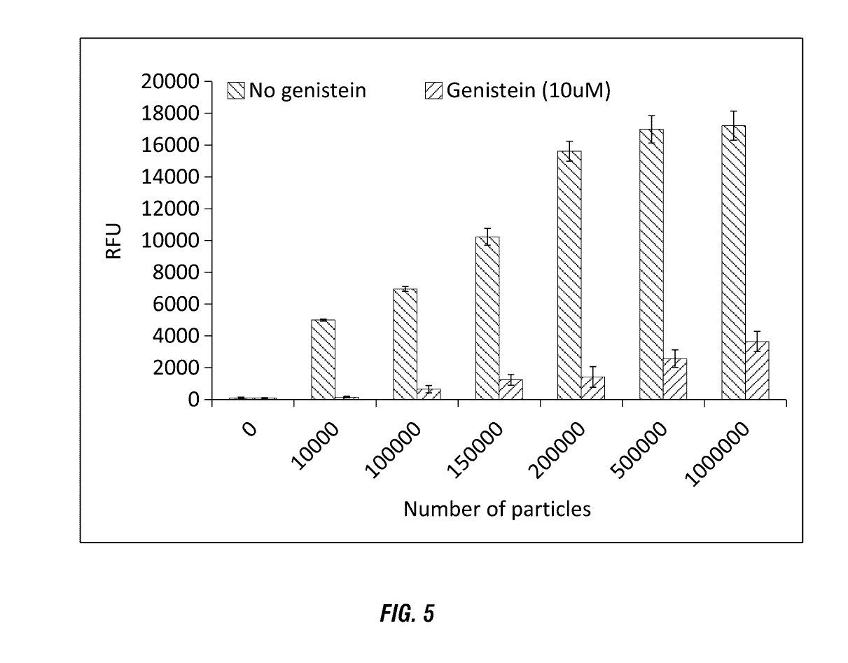

The surface of RBCs is high in the glucose transporter GLUT1. Modification of nanoparticles with glucose allows the nanoparticles to bind to RBCs in order to enhance the circulation of the nanoparticles, thereby enhancing the efficacy of nanoparticle delivery. Model nanoparticles composed of fluorescent polystyrene were unmodified or surface modified with fructose, glucose, or polyethylene glycol (PEG). As can be seen in FIG. 2, modification of nanoparticles results in a slightly positive zeta-potential (i.e. electrostatic repulsion forces between the particles) and a very similar size. The nanoparticles that are modified with glucose bind to RBCs in vitro, and the binding has an apparent saturation point. (FIG. 3). The in vitro binding of glucose modified nanoparticles to RBCs was maintained in the presence of increasing amounts of serum, demonstrating that serum components do not interfere with the RBC-particle interaction. (FIG. 4). Further, treatment of RBCs with genistein (a competitive GLUT1 inhibitor) prevents interaction with glucose modified nanoparticle, demonstrating that binding of glucose modified nanoparticles is specifically mediated by GLUT1. (FIG. 5).

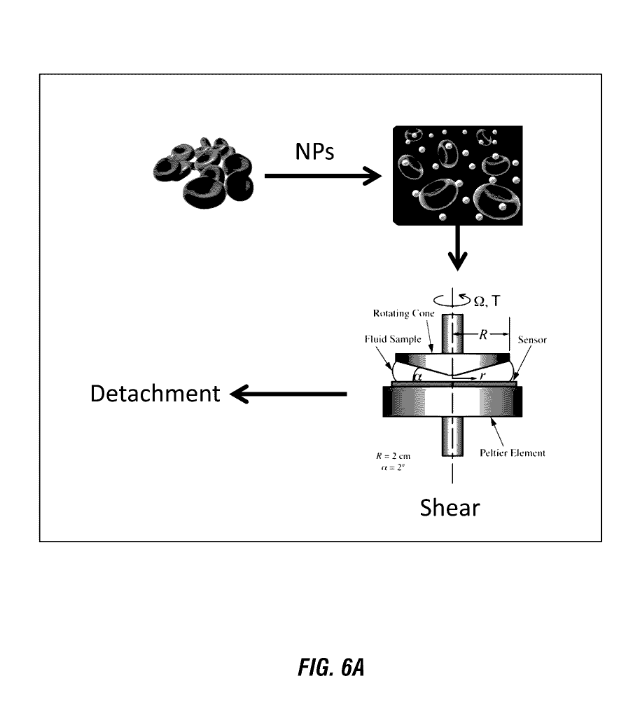

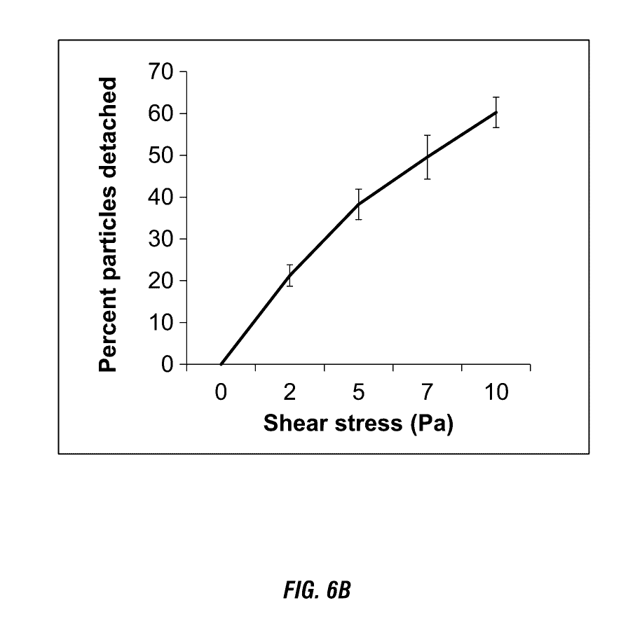

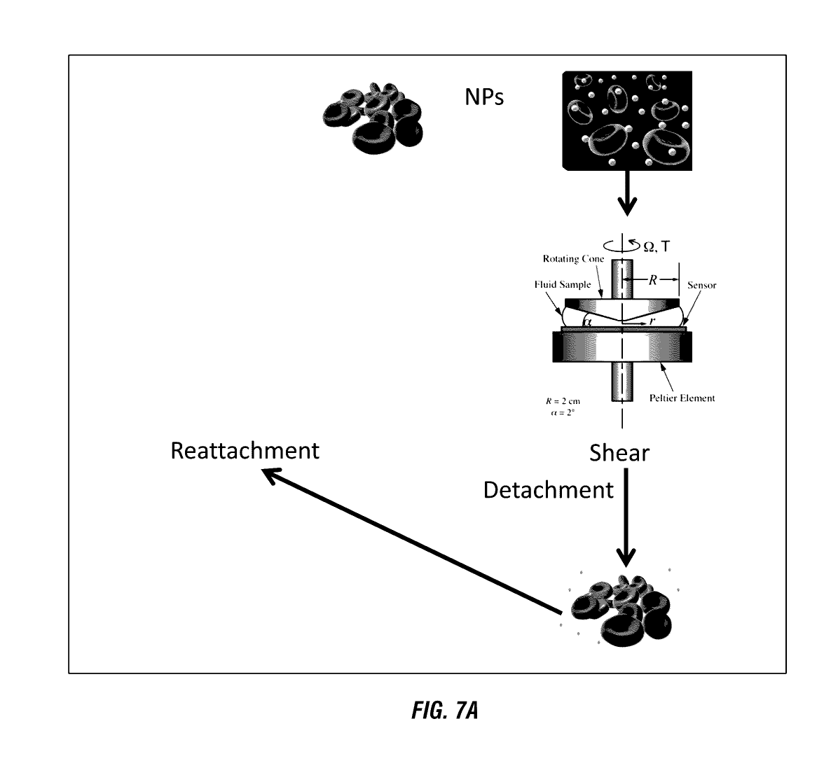

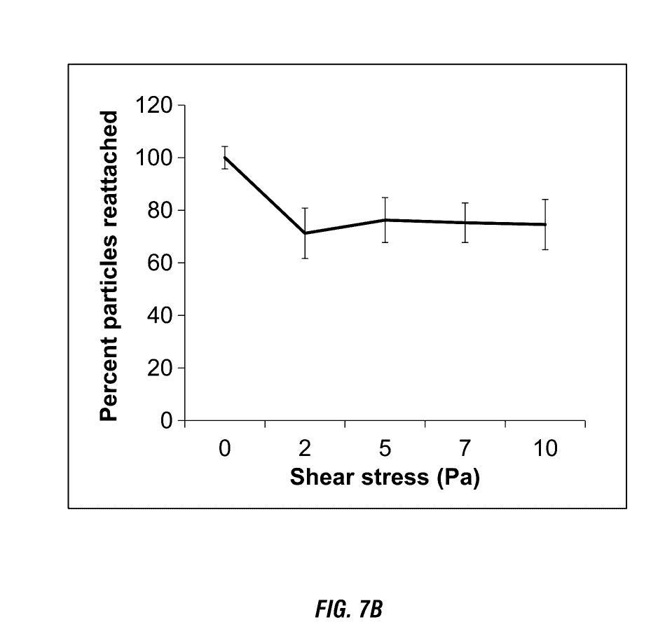

To determine whether the attachment of glucose modified nanoparticles is reversible, RBCs with glucose modified nanoparticles bound were subjected to shear stress. As shown in FIG. 6, the binding of glucose modified nanoparticles is reversible under shear stress. However, the detachment of the nanoparticles was not due either to loss of glucose modification of the nanoparticles, or to loss of GLUT1 from the surface of RBCs because glucose-modified nanoparticles were able to reattach to RBCs after shear stress. (FIG. 7).

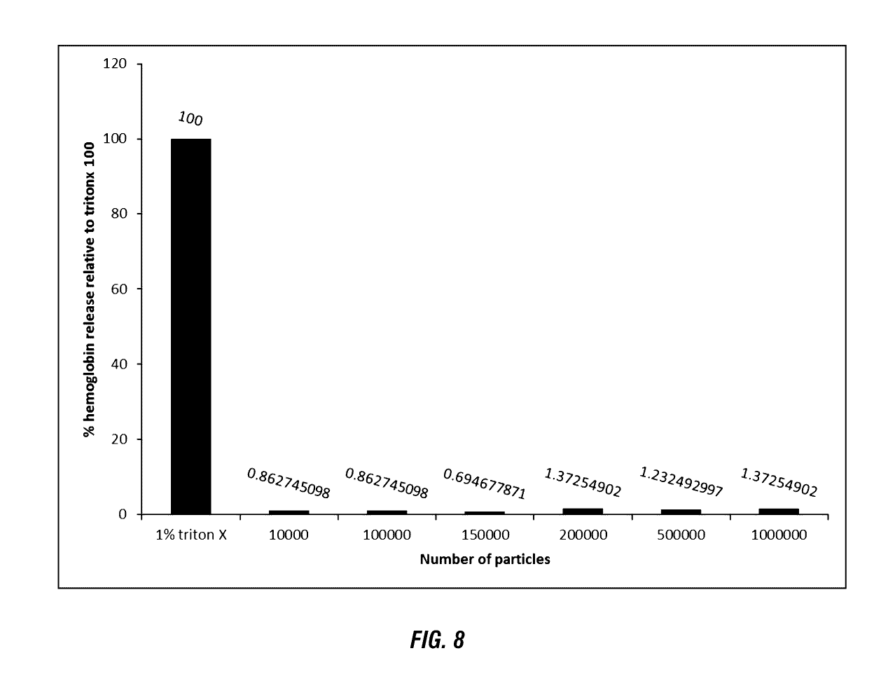

The toxicity of the glucose modified nanoparticles to RBCs we examined by determining the percentage of hemoglobin released by RBCs exposed for 24 hours to increasing numbers of nanoparticles, compared to lysis with 1% triton X detergent. As shown in FIG. 8, do significant hematoxicity was detected at any level of nanoparticle exposure.

These results demonstrate that surface molecules of RBCs represent a useful target for non-covalent attachment of modified nanoparticles. In the case of glucose modified nanoparticles, the abundance of GLUT1 provides a target-rich environment for binding, and the interaction is specific and able to occur in the presence of serum. Further, the non-covalent nature of the interaction allows detachment of the nanoparticles from the surface of the RBCs without damage to either the modified nanoparticle or the RBC.

### Example 2

### Glucose Modified Nanoparticles are Biodistributed In Vivo

The efficacy of nanoparticle modification for attachment to blood cells is demonstrated above. Biodistribution and pharmacokinetic characteristics were determined for in vivo applications were determined to assess applicability of enhanced nanoparticle delivery. Pharmacokinetic modeling of the unmodified or surface modified nanoparticles was carried out using the following equations:

$$\begin{matrix}ln Cp = ln {Cp}_{0} - {k}_{el} \times t & Equation 1 \\ {k}_{el} = \frac{( ln {Cp}_{0} - ln {Cp}_{t} )}{t} & Equation 2 \\ {Cp}_{0} = \frac{Dose}{Vd} & Equation 3\end{matrix}$$

The calculated elimination rate constant (kₑₗ) and circulation half time (T_{1/2}) are shown in Table 1 below. As demonstrated in Table 1, glucose modified nanoparticles exhibit better pharmacokinetic properties than the other particles, including PEG-modified nanoparticles.

TABLE 1
Pharmacokinetic parameters of nanoparticles.

| | | Fructose | Glucose | PEG 2K |
|---|---|---|---|---|
| Parameters | Control beads | beads | beads | beads |
| kₑₗ (Elimination | 0.0871 ± | 0.0885 ± | 0.00551 ± | 0.00914 ± |
| rate constant) | 0.0045/hr | 0.0059/hr | 0.00054/hr | 0.00074/hr |
| T_{1/2} | 7.96 ± | 7.84 ± | 126 ± | 81.26 ± |
| | 1.52 mins | 1.85 mins | 12.56 mins | 3.12 mins |

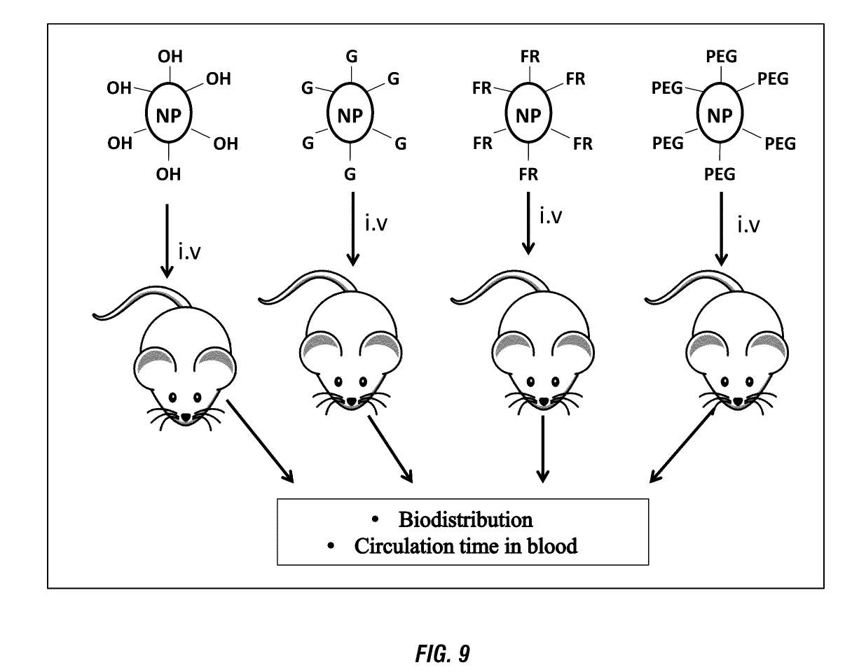

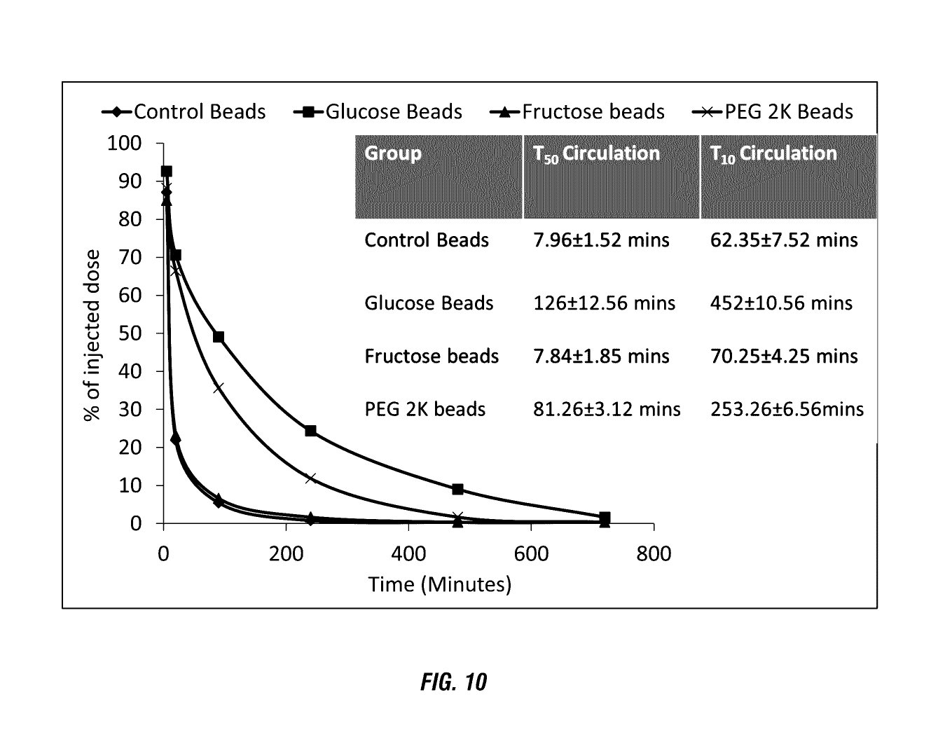

In order to determine whether the modified nanoparticles could be effectively circulated and distributed in vivo, the same modified nanoparticles were administered intravenously (i.v.) to BALB/c mice. (See FIG. 9). The amount of time each type of particle circulated in the mice was determined by collecting blood from mice at various times after administration of the particles and detecting the percentage of the injected dose remaining. As shown in FIG. 10, nanoparticles modified with glucose exhibited the best circulation profile, compared to unmodified particles, particles modified with fructose, or particles modified with PEG.

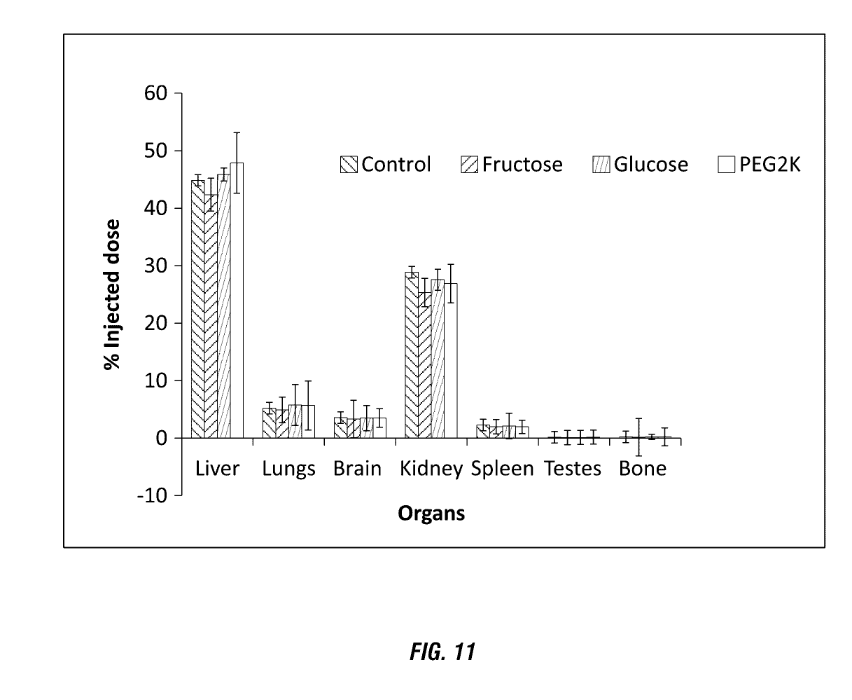

The distribution of the nanoparticles in various tissues of mice to which unmodified or modified nanoparticles were administered was determined after 24 hours. As shown in FIG. 11, the glucose modified nanoparticles exhibited a tissue distribution similar to that of other nanoparticles, including PEG-modified nanoparticles.

The above specification provides a description of various surface-modified particles and various methods of generating and using surface-modified particles to enhanced delivery in vivo. Since many embodiments can be made without departing from the spirit and scope of the invention, the invention resides in the claims.

1: A method of enhancing efficiency of in vivo particle delivery comprising:
conjugating a molecule to a particle to produce a surface modified particle, wherein said molecule mediates binding to a protein, peptide, lipid or carbohydrate on the surface of a blood cell.

2: The method of claim 1 wherein said molecule binds to a protein or peptide on the surface of a blood cell.

3: The method of claim 2 wherein said binding to a protein or peptide is reversible.

4: The method of claim 3 wherein said molecule conjugated to said particle is a non-toxic small molecule.

5: The method of claim 3 wherein the protein or peptide is selected from the group consisting of receptors, adhesion molecules, enzymes, structural molecules, channels, and transporter proteins.

6: The method of claim 3 wherein said protein or peptide is a GLUT1 transporter protein.

7: The method of claim 4 wherein the non-toxic small molecule is glucose.

8: The method of claim 2 where the molecule is covalently conjugated to the particle.

9: The method of claim 2 wherein the molecule is non-covalently conjugated to the particle.

10: The method of claim 1 wherein the particle is a nanocarrier or nanoparticle or micron size particle.

11: The method of claim 1 wherein said particle is selected from the group consisting of nanoshells, liposomes, polymeric carriers, protein carriers, carbohydrate carriers, neosomes, dendrimers, micells, carbon nanotubes, semiconductor or metal particles, and polymers-conjugate drug constructs.

12: The method of claim 1 wherein said blood cell is a red blood cell.

13: The method of claim 1 wherein said blood cell is a leukocyte or platelet.

14: A modified microscale or nanoscale carrier assembly comprising:
a particle; and
a molecule conjugated to the surface of said particle, wherein said molecule reversibly binds to a protein or peptide on the surface of a blood cell.

15: The modified carrier assembly of claim 14 wherein said particle is a selected from the group consisting of nanoshells, liposomes, polymeric carriers, protein carriers, carbohydrate carriers, neosomes, dendrimers, micells, carbon nanotubes, semiconductor or metal particles, and polymers-conjugate drug constructs.

16: The modified assembly of claim 14 wherein said blood cell is a red blood cell.

17: The modified assembly of claim 14 wherein said molecule conjugated to said particle reversibly binds to a molecule on the surface of a blood cell.

18: The modified carrier assembly of claim 14 wherein said molecule conjugated to said particle binds to a GLUT1 transporter.

19: The modified assembly of claim 18 wherein said molecule conjugated to said particle is glucose.

20: The modified assembly of claim 14 wherein said conjugation of said molecule to said particle is non-covalent.

21: A method of diagnosing or treating a disease comprising:
administering to an individual a modified microscale or nanoscale carrier assembly comprising:
a microscale or nanoscale particle; and
a molecule conjugated to the surface of said particle, Wherein said molecule reversibly binds to a protein or peptide on the surface of a blood cell.

22: The method of claim 21 wherein said molecule conjugated to the surface of said particle binds to a molecule on the surface of a red blood cell.

23: The method of claim 21 wherein the elimination rate constant (kel) for said modified microscale or nanoscale carrier assembly is less than 0.009/hr.

24: The method of claim 21 wherein the circulation half time (T_{1/2}) for said modified microscale or nanoscale carrier assembly is greater than 85 minutes.

25: The method of claim 21 wherein said administering comprises intravenous injection.

26: The method of claim 21 wherein said administering comprises obtaining a blood sample from said individual, combining said modified microscale or nanoscale carrier assembly with said blood sample, and introducing said blood sample with said modified microscale or nanoscale carrier assembly to the individual.
